(19) United States
(12) Patent Application Publication
(10) Pub. No.: US 2022/0005579 A1
(43) Pub. Date: Jan. 6, 2022

(54) SUPPLEMENT PROPOSAL SYSTEM

(71) Applicant: FINC TECHNOLOGIES INC., Tokyo (JP)

(72) Inventors: Yosuke SUZUKI, Tokyo (JP); Akiko MIYAZAKI, Tokyo (JP)

(21) Appl. No.: 17/280,713

(22) PCT Filed: Aug. 26, 2019
(86) PCT No.: PCT/JP2019/033251

§ 371 Date: Mar. 26, 2021

(30) Foreign Application Priority Data
Sep. 29, 2018 (JP) 2018-186222

(51) Int. Cl. G16H 20/60; G16H 10/20; G16H 50/70; G16H 10/60

(52) U.S. Cl. CPC G16H 20/60; G16H 10/20; G16H 20/30; G16H 10/60; G16H 50/70

(57) ABSTRACT

A supplement tailored to a user. This supplement proposal system includes: a supplement information storage unit for storing nutrients contained in a supplement, an answer reception unit for receiving an answer from a user to questions about lifestyle habit, an insufficient nutrient identification unit for identifying nutrients lacking in the user in response to attribution of the user and the answer from the user, a supplement selection unit for selecting the supplements including the insufficient nutrients, and a supplement proposal unit for proposing the selected supplements to the user.

### TECHNICAL FIELD

The present disclosure relates to supplement proposal system.

### BACKGROUND ART

Supplements are sometimes used as a nutrition supplement ingested from a meal. For example, Patent Literature 1 discloses a system for notifying supplements based on nutrients ingested at a restaurant.

### PRIOR ART

### Patent Literature

Patent Literature 1: Japanese Patent No. 6112328

### SUMMARY OF THE INVENTION

### Technical Problem

However, the system described in Patent Literature 1 has to be linked with an ordering system at a restaurant, which is troublesome.

The present disclosure has been made in view of such a background, and an object of the present disclosure is to provide a technique that can easily propose a supplement suitable for a user.

### Technical Solution

The main invention of the present disclosure for achieving the above object is a supplement proposal system including: a supplement information storage unit for storing nutrients contained in a supplement, an answer reception unit for receiving an answer from a user to questions about lifestyle habit, an insufficient nutrient identifying unit for identifying nutrients lacking in the user in response to attribution of the user and the answer from the user, a supplement selection unit for selecting the supplements including the insufficient nutrients, and a supplement proposal unit for proposing the selected supplements to the user.

Other problems disclosed in the present application and technical solutions thereof will be described in the embodiments of the invention and the accompanying figures.

### Advantageous Effects

According to the present disclosure, it is possible to propose a supplement suitable for a user.

### BRIEF DESCRIPTION OF THE DRAWING

FIG. 1 is a diagram showing an example of an overall configuration of a supplement proposal system according to one embodiment of the present disclosure.

FIG. 2 is a diagram showing an example of a hardware configuration of the management server 1.

FIG. 3 is a diagram showing an example of a software configuration of the management server 1.

FIG. 4 is a figure showing an example of a configuration of lifestyle habit information stored in the lifestyle habit information storage unit 151.

FIG. 5 is a figure showing an example of a configuration of supplement information stored in the supplement information storage unit 152.

FIG. 6 is a diagram showing an example of a configuration of answer information stored in the answer storage unit 153.

FIG. 7 is a diagram illustrating a process executed by the management server 1.

FIG. 8 is a figure showing an example of a start screen for starting a question.

FIG. 9 is a figure showing an example of a screen for inquiring about basic information of a user.

FIG. 10 is a figure showing an example of a screen for inquiring about basic information of a user.

FIG. 11 is a figure showing an example of a screen for inquiring about a user's troubles.

FIG. 12 is a figure showing an example of a screen for inquiring about a user's eating habits.

FIG. 13 is a figure showing an example of a screen for inquiring about a user's exercise habits.

FIG. 14 is a figure showing an example of a screen for inquiring about a user's drinking habit.

FIG. 15 is a figure showing an example of a screen for inquiring about a user's allergy.

FIG. 16 is a figure showing an example of a screen for inquiring about a user's allergy.

FIG. 17 is a figure showing an example of a display of a supplement proposed to a user.

FIG. 18 is a figure showing an example of a screen for displaying a detailed description of a supplement proposed to a user.

### DETAILED DESCRIPTION OF THE EMBODIMENTS

The contents of the embodiment of the present disclosure will be listed and described. A supplement proposal system according to one embodiment of the present disclosure has the following configuration.

[Item 1]

A supplement proposal system including:

a supplement information storage unit for storing nutrients contained in a supplement,

an answer reception unit for receiving an answer from a user to questions about lifestyle habit,

an insufficient nutrient identifying unit for identifying nutrients lacking in the user in response to attribution of the user and the answer from the user,

a supplement selection unit for selecting the supplements including the insufficient nutrients, and

a supplement proposal unit for proposing the selected supplements to the user.

[Item 2]

The supplement proposal system as set forth in Item 1,

wherein the supplement information storage unit further stores a degree of recommendation of the supplement for the user's troubles,

the answer reception unit further receives an input of the degree of troubles from the user,

the supplement selection unit determines a degree of recommendation of the supplement according to the degree of recommendation corresponding to the received trouble and the degree of the received trouble, and selects a number of supplement in a predetermined range including the insufficient nutrients based on the degree of recommendation of the supplement.

[Item 3]

The supplement proposal system as set forth in Item 2,

wherein the supplement information storage unit stores a description of the supplement function against the troubles for each of the troubles with respect to the supplement, and

the supplement proposal unit outputs to the user a description of the supplement and the function corresponding to the received troubles, with the selected supplement.

[Item 4]

The supplement proposal system as set forth in any one of Items 1 to 3,

wherein the supplement information storage unit further stores a description of the supplement, and

the supplement proposal unit outputs the corresponding description to the user with the selected supplement.

[Item 5]

The supplement proposal system as set forth in any one of Items 1 to 4,

wherein the supplement information storage unit further stores allergy substances contained in the supplement,

the answer reception unit further receives an answer to the user's allergy, and

the supplement selection unit selects those not containing the allergy substances among the supplements containing the insufficient nutrients, in accordance with the answer regarding the allergy.

FIG. 1 is a diagram showing an example of an overall configuration of a supplement proposal system according to one embodiment of the present disclosure. The supplement proposal system of the present embodiment includes a management server 1. The management sever 1 is communicably connected to a user terminal 2 via a communication network 3. The communication network 3 is, for example, the Internet, and is constructed by a public telephone line network, a mobile phone line network, a wireless communication path, Ethernet (registered trademark), or the like.

The supplement proposal system of the present embodiment is intended to propose a supplement (also referred to as a nutrition supplement or a health supplement) to a user. The supplement proposal system of the present embodiment gives a question to the user and proposes a supplement in accordance with the answer. The questions include questions about user's lifestyle habits, and questions that inquire about user's troubles (problems that users are aware of, that is, needs) such as diet or skin anti-aging, fatigue recovery, muscle strength improvement, stress, and concentration power. In the present embodiment, the questions about lifestyle habits include questions about eating habits, questions about exercise, and questions about preferences such as drinking and smoking, and the supplement suggestion system estimates nutrients lacking in the user in accordance with the answers to these questions. In the supplement proposal system of the present embodiment, among the supplements for supplementing insufficient nutrients, those that are expected to solve the user's troubles are preferentially selected and proposed to the user.

The management server 1 is a computer that proposes supplements in response to questions for a user. The management server 1 is, for example, a personal computer, a workstation, or a virtual computer using cloud computing. The management server 1 transmits a question to the user terminal 2, receives an answer transmitted from the user terminal 2, and transmits information about the supplement proposed according to the answer to the user terminal 2. It is assumed that the management server 1 executes these functions as a so-called Web application.

The user terminal 2 is a computer such as, for example, a mobile phone, a smartphone, a tablet computer, or a personal computer which is operated by the user. The user operates the user terminal 2 to access the management server 1 and acquire a question. The user terminal 2 displays the question to receive t input of the answer from the user, transmits the answer to the management server 1, and displays information on the recommended supplement that is responded thereto.

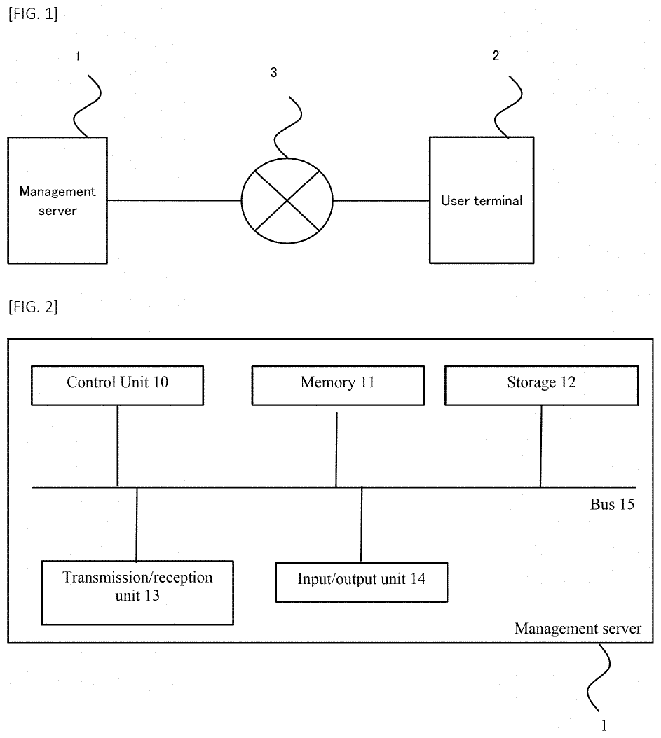

FIG. 2 is a diagram showing an example of a hardware configuration of the management server 1. The management server 1 includes at least a control unit 10, a memory 11, a storage 12, a transmission/reception unit 13, an input/output unit 14, and the like, which are electrically connected to each other through a bus 15. The control unit 10 is an arithmetic device that controls the operation of the entire management server 1, controls the transmission and reception of data between each element, and performs information processing necessary for application execution and authentication processing. For example, the control unit 10 is a CPU (Central Processing Unit), and executes each information processing by executing a program or the like stored in the storage 12 and deployed in the memory 11. The memory 11 includes a main memory composed of a volatile storage device such as a DRAM (Dynamic Random Access Memory) and the like, and an auxiliary memory composed of a non-volatile storage device such as a flash memory or an HDD (Hard Disk Drive) and the like. The memory 11 is used as a work area or the like of the processor 10, and also stores a BIOS (Basic Input/Output System) executed when the management server 1 is started, various setting information, and the like. The storage 12 stores various programs such as application programs. A database (not shown) storing data used for each processing may be built in the storage 12. The transmission/reception unit 13 connects the management server 1 to the network. The transmission/reception unit 13 may be provided with a short-range communication interface such as Bluetooth (registered trademark) or Wi-Fi (registered trademark). The input/output unit 14 is an information input device such as a keyboard and a mouse and the like, and an output device such as a display and the like. The bus 15 is commonly connected to each of the above elements and transmits, for example, an address signal, a data signal, and various control signals.

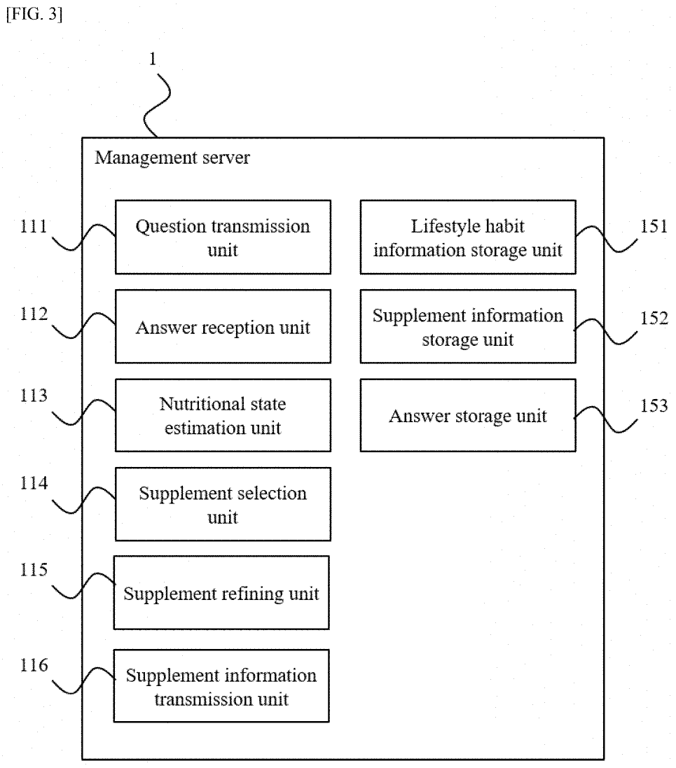

FIG. 3 is a diagram showing an example of a software configuration of the management server 1. The management server 1 includes a question transmission unit 111, an answer reception unit 112, a nutritional state estimation unit 113, a supplement selection unit 114, a supplement refining unit 115, a supplement information transmission unit 116, a lifestyle habit information storage unit 151, a supplement information storage unit 152, and an answer storage unit 153.

Further, the question transmission unit 111, the answer reception unit 112, the nutritional state estimation unit 113, the supplement selection unit 114, the supplement refining unit 115, and the supplement information transmission unit 116 are realized by allowing the control unit 10 included in the management server 1 to readout the program stored in the storage 12 to the memory 11 and execute it, and the lifestyle habit information storage unit 151, the supplement information storage unit 152, and the answer storage unit 153 are realized as a part of the storage area provided by at least one of the memory 11 and the storage 12.

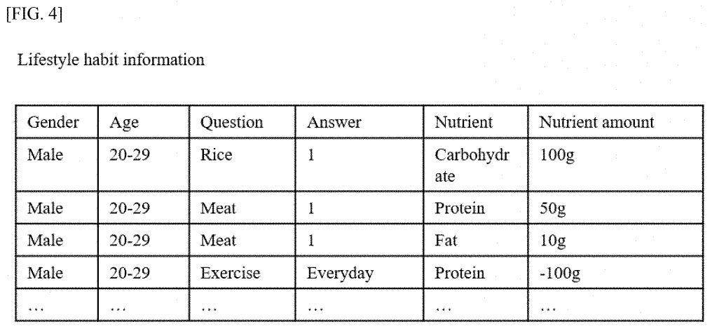

The lifestyle habit information storage unit 151 stores information (hereinafter referred to as lifestyle habit information) for estimating the nutrients ingested by the user based on the answers to the questions regarding the lifestyle habit. FIG. 4 is a figure showing an example of a configuration of lifestyle habit information stored in the lifestyle habit information storage unit 151. The lifestyle habit information includes nutrients and nutrient amounts in association with gender, age, the questions and the answers. For the gender and age, conditions for the user's gender and age are set. The question includes information that identifies a question about lifestyle habits that inquires of the user. The answer includes the conditions for the answer to the question. The nutrients are information for identifying the nutrients that the user is ingesting, and the nutrient amount is the quantity of this nutrient. That is, when the answer to the above question received from the user satisfying above gender and age conditions satisfies the conditions set in an answer column, it indicates that the nutrient indicated by a nutrient column is ingested the user by the amount indicated by a nutrient amount column. For example, in the example of FIG. 4, when the answer to the question indicated by “rice” (for example, “how many bowls of rice are consumed per day in a bowl?”) is “1 (bowl)”, it can be estimated that the user is ingesting “100 g” of carbohydrates. Similarly, when the answer to the question indicated by “meat” (for example, question “‘how many pieces of loin meat do you eat per day?’) is “1 (pieces)”, it can be estimated that the user is ingesting 50 g of protein and 10 g of fat. Similarly, the amount of nutrients ingested by the user can be estimated based on the attributes of the user and the lifestyle habit information corresponding to the answer from the user.

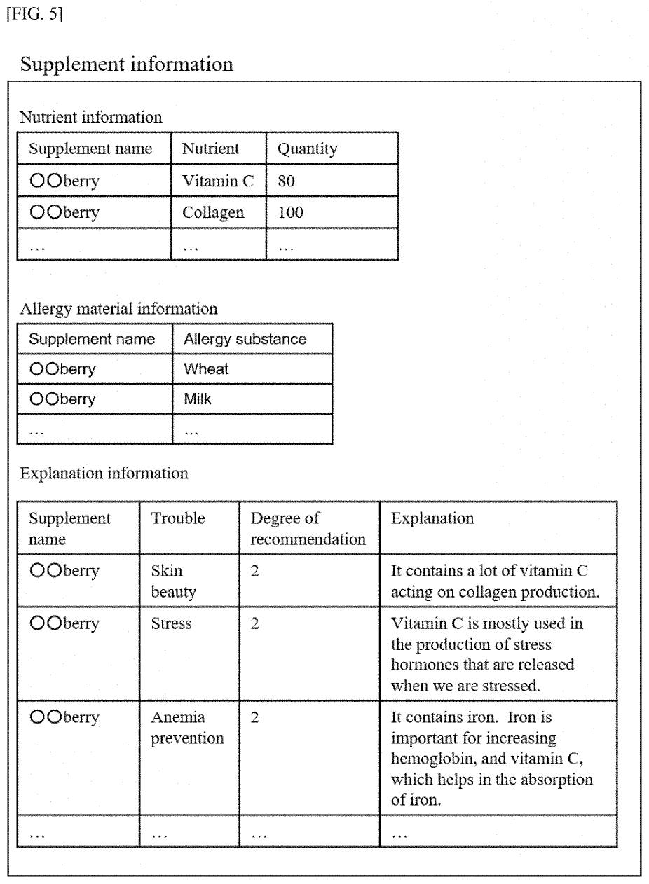

The supplement information storage unit 152 stores information about the supplement (hereinafter, referred to as supplement information). FIG. 5 is a figure showing an example of a configuration of supplement information stored in the supplement information storage unit 152. As shown in the figure, the supplement information includes nutrient information, allergy substance information and explanation information. The nutrient information includes the nutrients contained in the supplement and their quantity in association with the supplement name. The allergy substance information includes allergy substances contained in the supplement in association with the supplement name. The explanation information includes a degree of recommendation and an explanation regarding the troubles that can be solved or improved by the supplement in association with the supplement name. The description in this explanation column is a description of what function the supplement has for troubles.

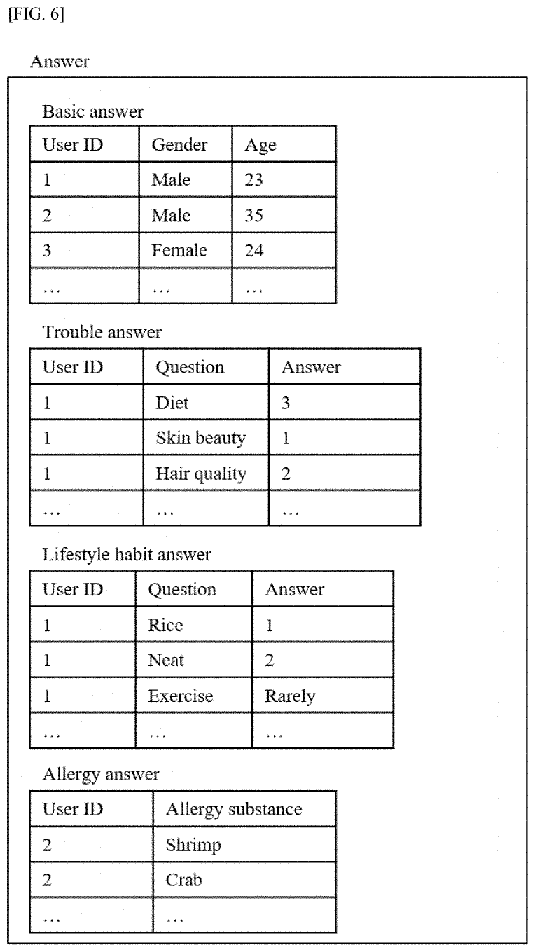

The answer storage unit 153 stores information including an answer from the user (hereinafter, referred to as answer information). FIG. 6 is a figure showing an example of a configuration of answer information stored in the answer storage unit 153. As shown in the figure, the answer information includes an answer about the user's basic information (basic answer), an answer about troubles (trouble answer), a lifestyle habit-related answer (lifestyle habit answer), and an answer about allergy substances (allergy answer). In the basic answer, gender and age are included in association with the user ID indicating the user. In the trouble answer, the answer to each question is stored in association with the user ID. The answer in the trouble answer is a value indicating the intensity of the troubles related to the question (for example, it can be made in three stages of 1 to 3). In the lifestyle habit answer, the answer to each question is stored in association with the user ID. The response to the lifestyle habit answer sets various values such as the amount of foods ingested, the frequency of exercise, and the amount of drinking. The allergy answer includes an allergy substance to which the user is allergic, in association with the user ID. Further, for a user who does not have allergies, an allergy answer is not registered.

The question transmission unit 111 transmits a question to the user. The question includes a question inquiring about the basic information of the user (in this embodiment, it is assumed to be gender and age), a question about user's lifestyle habits, and a question inquiring about the user's troubles (problems that the user is aware of, that is, needs). The question transmission unit 111 can transmit a question to the user terminal 2 in the form of, for example, HTML.

The answer reception unit 112 receives an answer to the question from the user. The answer reception unit 112 registers the received answer in the answer storage unit 153.

The nutritional state estimation unit 113 identifies nutrients lacking in the user based on the answer from the user. In the present embodiment, the nutritional state estimation unit 113 can read the basic answer and the lifestyle habit answer corresponding to the user ID from the answer storage unit 153, retrieve the lifestyle habit information corresponding to the read basic answer and lifestyle habit answer from the lifestyle habit information storage unit 151, sum up the amount of nutrients in the retrieved lifestyle habit information for each nutrient, and estimate the nutrients ingested by the user. The nutritional state estimation unit 113 also estimates the amount of nutrients that the user should consume (hereinafter referred to as the ideal nutrient amount, which is set in advance. The ideal nutrient amount may be stored in the ideal nutrient amount storage unit, which stores the ideal nutrient amount in relation to the basic information of gender and age). The amount of insufficient nutrients can be calculated by subtracting the intake amount from the ideal nutrient amount.

The supplement selection unit 114 selects a supplement for supplementing the nutrition lacking in the user. The supplement selection unit 114 can select a supplement containing an insufficient nutrient from the supplement information storage unit 152. Further, the supplement selection unit 114 may select a supplement according to the amount of supplementing the insufficient nutrients.

The supplement refining unit 115 refines supplements according to the user's troubles. The supplement refining 115 can refine the supplements according to the degree of recommendation corresponding to the user's troubles, with reference to the explanation information included in the supplement information.

The supplement information transmission unit 116 transmits the supplement to the user. The supplement information transmission unit 116 can read the explanation information from the supplement information storage unit 152 and transmit the supplement refined by the supplement refining unit 115 to the user terminal 2.

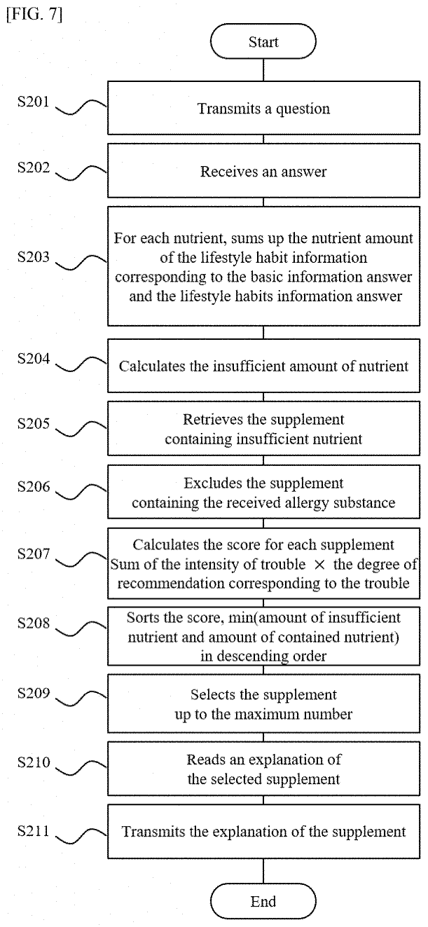

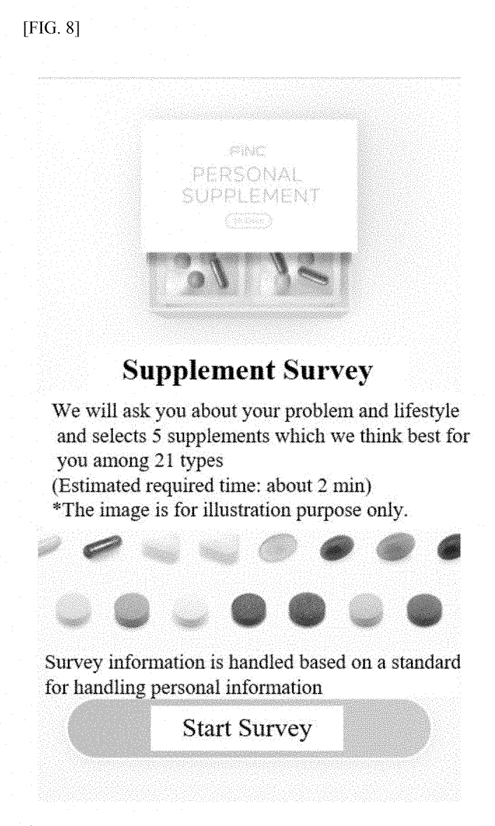

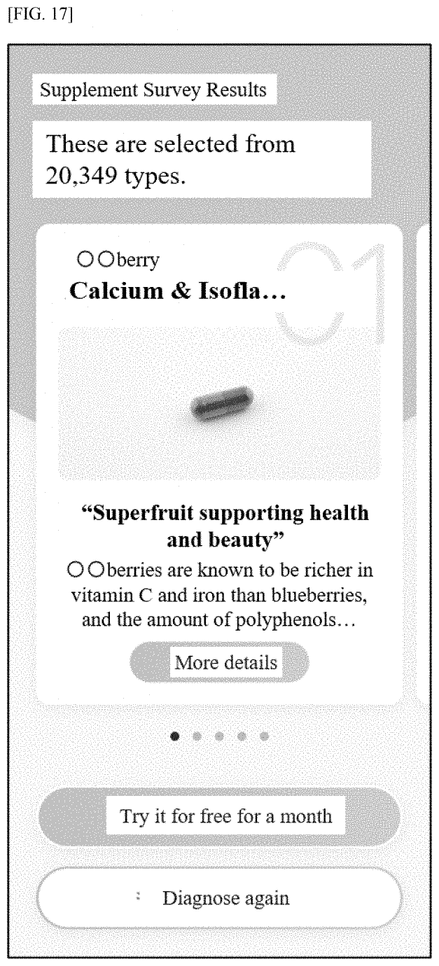

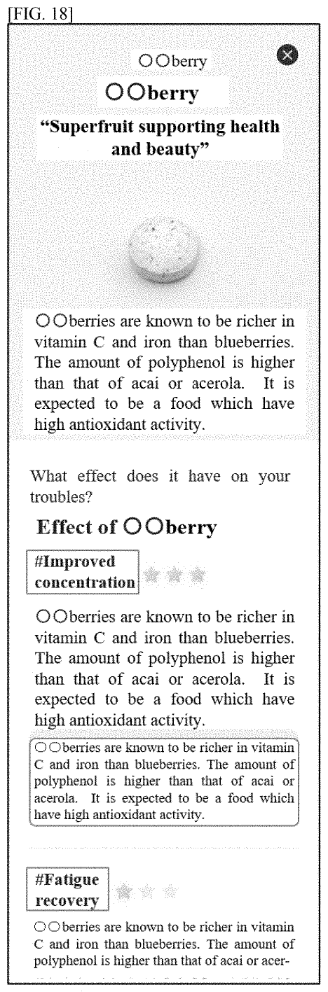

FIG. 7 is a diagram illustrating a process executed by the management server 1. Further, FIGS. 8 to 16 are figures showing examples of an answer reception screen displayed on the user terminal 2. FIGS. 17 and 18 are figures showing display examples of a supplement proposed to the user.

When the question transmission unit 111 transmits a question to the user from the management server 1 (S201), the user terminal 1 receives an answer to a question. FIG. 8 is a figure showing an example of a start screen for starting a question. The question is started by pressing the “start survey” button on the start screen. As described above, the question includes a question inquiring about the basic information of the user, a question about user's lifestyle habits, and a question inquiring about the user's troubles.

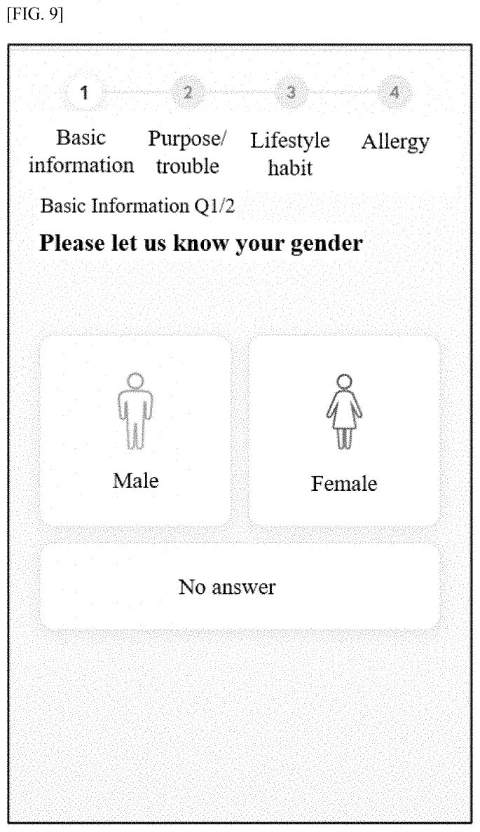

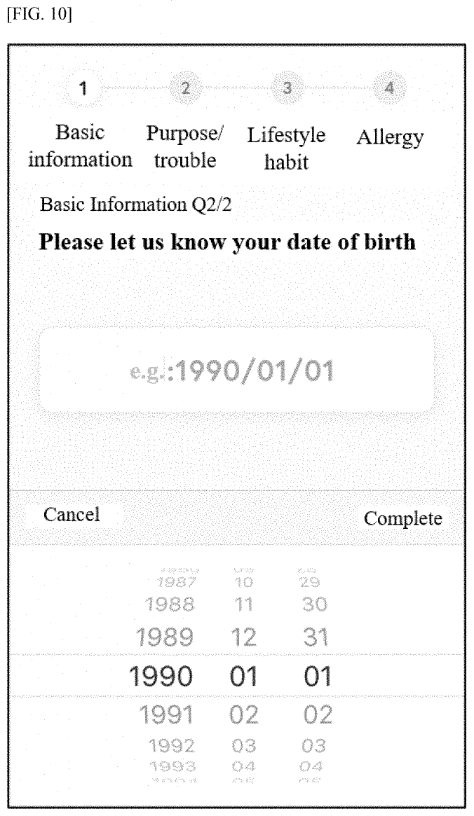

FIGS. 9 and 10 are figures showing screen examples for inquiring about basic information of a user. In the example of FIG. 9, a question is given about the gender of the user, and in the example of FIG. 10, a question is given about the age of the user. The user terminal 1 may calculate the age of the user from the input date of birth, or may answer the date of birth to the management server 1 and calculate the age on the management server 1 side.

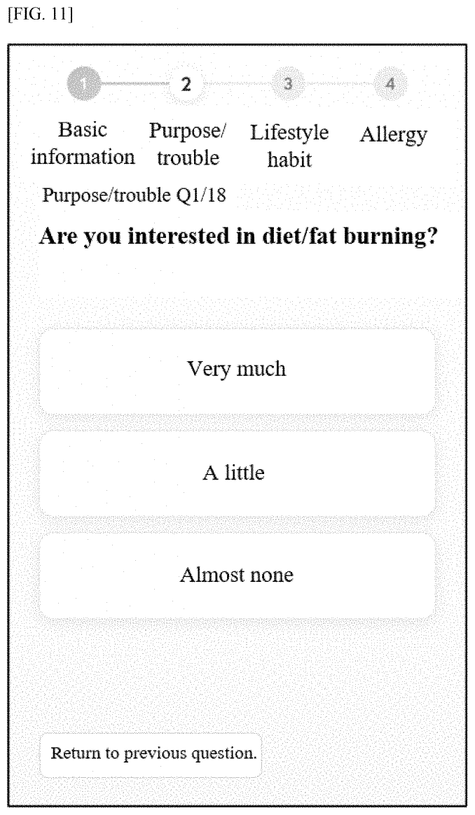

FIG. 11 is a figure showing an example of a screen for inquiring about the user's troubles. In the example of FIG. 11, a question is given about the degree of interest in “diet/fat burning”. In the option, the degree of troubles is selected as “very much”, “a little”, and “almost none”. Although FIG. 11 shows only questions about “diet/fat burning”, questions are given even regarding the troubles such as, diet/fat burning, skin beauty/skin anti-aging, hair quality improvement, muscle strength/sports performance, fatigue recovery, stress, insight/concentration power, preventing anemia, eye fatigue, bowel movement, swelling, body coldness, rough skin, menstrual pain/menstrual irregularities, body less prone to catching colds, poorly sleeping, healthy teeth/bones, blood pressure, blood sugar level, triglyceride/cholesterol, etc.

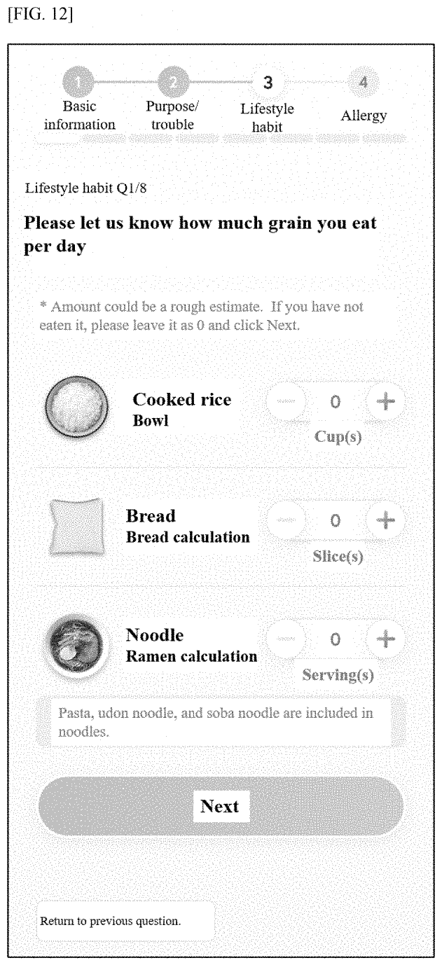

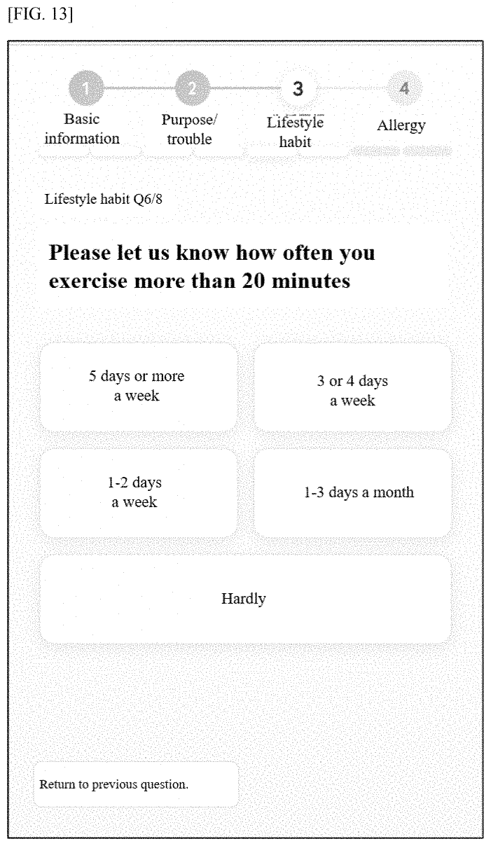

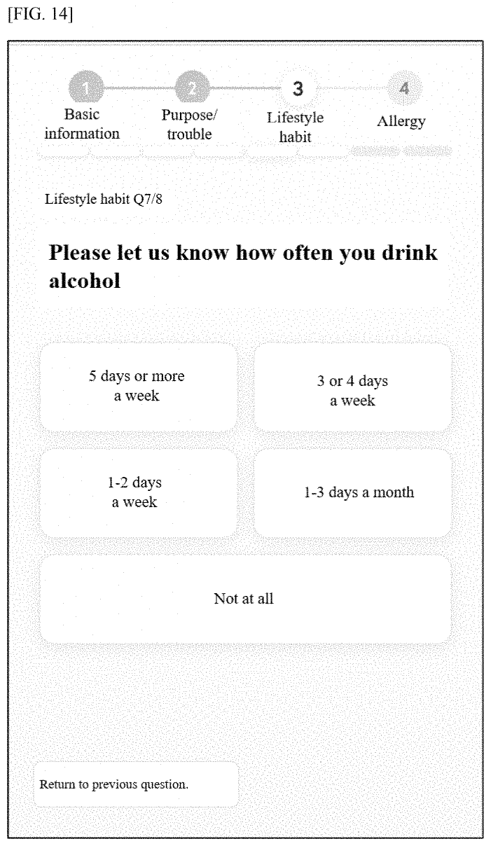

FIGS. 12 to 14 are figures showing screen examples for inquiring about the lifestyle habits of the user. In FIG. 12, a question is given about the meal that the user is ingesting. In FIG. 13, a question is given about the exercise habit of the user (in the example of FIG. 13, the exercise frequency at a predetermined intensity). In FIG. 14, a question is given about the user's preference (frequency of drinking alcohol in the example of FIG. 14).

The answer input in the screen example as described above is transmitted from the user terminal 2 to the management server 1, the answer reception unit 112 of the management server 1 receives it (S202), and the received answer is registered in the answer storage unit 153. The nutritional state estimation unit 113 reads the basic answer and the lifestyle habit answer corresponding to the user who received the answer from the answer storage unit 153, and thus reads the lifestyle habit information corresponding to the basic answer and the lifestyle habit answer from the lifestyle habit information storage unit 151, and sums up nutrient amounts of the read lifestyle habit information for each nutrient (S203). As described above, in response to the questions shown in FIGS. 12 to 14, it is possible to estimate the nutrients that the user is ingesting, according to the answers regarding the dietary habits that users are ingesting, the exercise habits, and the alcohol drinking habits. Next, the nutritional state estimation unit 113 calculates the insufficient amount of nutrients by subtracting the total amount of nutrients from the given ideal amount of nutrients for each nutrient (S204). The supplement selection unit 114 retrieves the supplements containing insufficient nutrients from a supplement information storage unit 152 (S205).

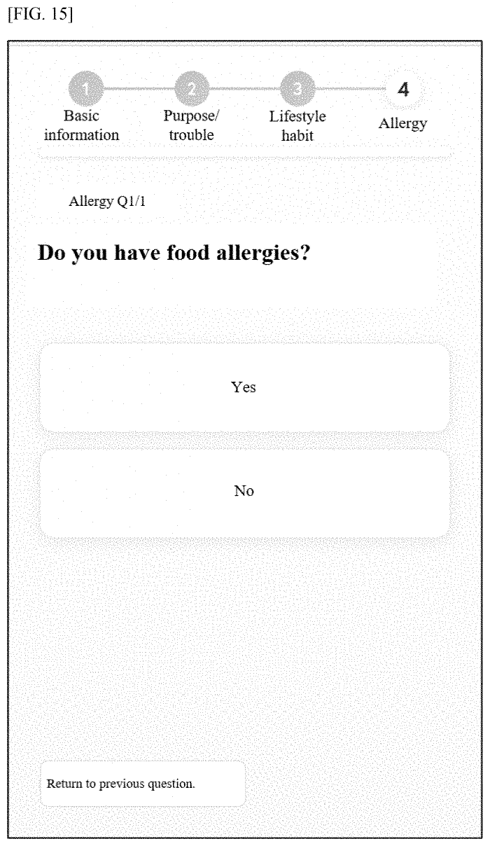

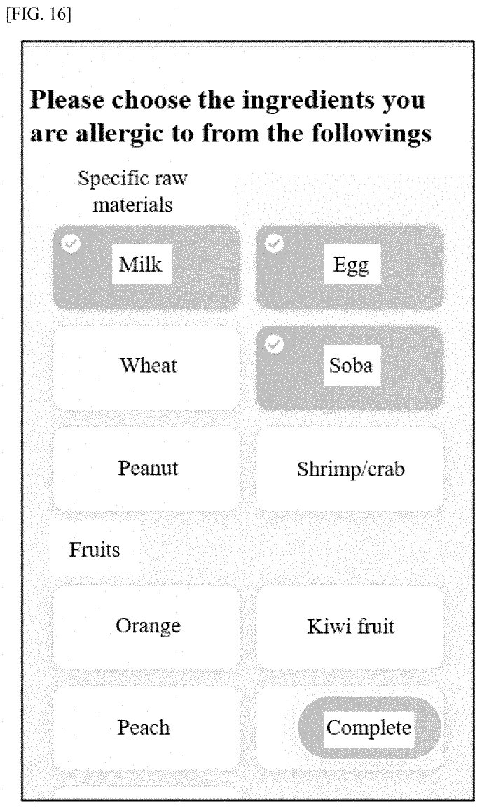

FIGS. 15 and 16 are figures showing screen examples for inquiring about a user's allergy. In FIG. 15, the presence or absence of allergies is inquired, and if there is an allergy, a specific allergen is selected, as shown in FIG. 16.

The supplement refining unit 115 retrieves the supplements containing the answered allergy substance from the supplement information storage unit 152, and excludes the retrieved supplement from the retrieval results in step S205 (S206). Further, the supplement refining unit 115 reads from the supplement information storage unit 152 what corresponds to the trouble answer among the explanation information corresponding to each supplement, calculates a score by multiplying the degree of recommendation of the read explanation information by the intensity of troubles (S207), sorts the score and the smaller side of the insufficient nutrient amount or the insufficient nutrient content in descending order (S208), and select a predetermined maximum number of supplements (for example, any number such as 5, 7, 10 can be set) (S209).

In the above-described embodiment, it was processed so as to exclude the supplement containing the allergy substance that was answered.

However, for example, in accordance with the user's constitution (including the allergies), supplements that should avoid ingestion (or should not ingest) may be excluded.

Further, the supplements that should avoid ingestion (or should not ingest) may be excluded in accordance with other foods and drinks (meal content, medication content, other supplements). The combination with nutrients contained in the meal or active ingredients contained in medications, or active ingredients contained in other supplements can prevent the effect from being excessive, or the effect from not being obtained.

Furthermore, the degree of recommendation may be adjusted according to the user's consciousness, preferences, hobbies, tastes, and ideologies. For example, it can be more personalized for users who have the ideology of avoiding specific nutrients or who want to actively take ingredients derived from herbal drugs.

Supplements that should avoid ingestion (or should not ingest) may be excluded in accordance with the physical condition and life stage. For example, pregnant or lactating users may be subjected to process such as excluding certain nutrients.

The supplement information transmission unit 116 reads the description of the supplement selected in step S209 from the explanation information of the supplement information storage unit 151 (S210), and transmits the read description of the supplement to the user terminal 2 as a description of the supplement proposed to the user (S211).

FIG. 17 is a figure showing an example of a display of a supplement proposed to a user. In the example of FIG. 17, the selected supplement is displayed along with the total number of supplement combinations (exactly, “20,349” ways in the example of FIG. 17). Also, when the “more details” button is pressed, a description of the selected supplement is displayed, as shown in FIG. 18. In the example of FIG. 18, although the description regarding one supplement (◯◯ berry) is displayed, descriptions of all the suggested supplements are displayed. Further, in addition to a brief description of the supplement, a description of how the supplement can function with respect to troubles that the user is more interested in is also displayed.

Although the present embodiment has been described above, the above-described embodiment is merely an example for facilitating the understanding of the invention and should not be construed as limiting the invention. The present disclosure can make some modifications and improvements without departing from the spirit thereof, and the present disclosure includes an equivalent thereof.

For example, in the present embodiment, the management server 1 is assumed to have both a functional unit and a storage unit, but the present disclosure is not limited thereto. For example, the storage unit may be provided in the database server, or may access the database server.

Further, in the present embodiment, the questions about lifestyle habits are questions about eating habits, questions about exercise habits, and questions about drinking habits, but in addition to these, or in place of at least one of these, questions such as questions about sleeping habits and questions about smoking habits may be included.

Further, in the present embodiment, the questions about lifestyle-related habits include questions about eating habits, questions about exercise, and questions about preferences such as drinking, but may be only one of these, and may be only these two types of questions. To identify nutrients,

In addition, as a question about lifestyle habits, in addition to questions about eating habits, questions about exercise, and questions about preferences such as drinking or smoking, or instead of at least one of these questions, questions to estimate the nutrients that the user is ingesting or that the user is not ingesting, log data such as meal logs obtained from applications that record meals or by asking questions about meals, exercise logs obtained from workout support applications, exercise sensors, etc., sleep logs obtained from applications and sensors that observe a sleeping state can be included. Based on these questions and log data, the user's eating habits can be estimated, and the nutrients that are ingested or not ingested can be estimated from the estimated eating habits.

Further, in the present embodiment, the supplement is only proposed, but the proposed supplement may be sold. In this case, the management server 1 can receive the order information from the user terminal 2 and sell the supplement according to the received order information. In addition, the supplement can be sold as a package in which one tablet or one packet is combined.

### DESCRIPTION OF REFERENCE NUMERALS

1: management server
2: user terminal
3: communication network
11l: question transmission unit
112: answer reception unit
113: nutritional state estimation unit
114: supplement selection unit
115: supplement refining unit
116: supplement information transmission unit
151: lifestyle habit information storage unit
152: supplement information storage unit
153: answer memory unit

1. A supplement proposal system comprising:
a supplement information storage unit for storing nutrients contained in a supplement;
an answer reception unit for receiving an answer from a user to each of questions about meals ingested by the user and questions about the user's exercise habits;
an insufficient nutrient identifying unit for identifying nutrients lacking in the user in response to attribution of the user and the answer about each of the meal and the exercise habits from the user;
a supplement selection unit for selecting the supplements including the insufficient nutrients;
a supplement refining unit for refining the selected supplements; and
a supplement proposal unit for proposing the refined supplements to the user;
wherein the supplement information storage unit further stores a degree of recommendation of the supplement regarding the user's trouble,
wherein the answer reception unit further receives an input on the trouble and the degree of the trouble from the user, and
wherein the supplement refining unit reads the supplement corresponding to the trouble according to the degree of recommendation of the supplement, with reference to the supplement information storage unit, and also selects a predetermined range of numbers of supplements based on the degree of the trouble among the supplements containing the insufficient nutrients.

2. The supplement proposal system according to claim 1, further comprising a lifestyle habit information storage unit that stores information for identifying the amount of nutrients that the user can ingest in association with the content of the meal and the content of the exercise habit,
wherein the insufficient nutrient identifying unit reads information for identifying the amount of the nutrients corresponding to the answer from the lifestyle habit information storage unit, and identifies the insufficient nutrient based on the read information.

3. The supplement proposal system according to claim 2,
wherein the lifestyle habit information storage unit stores information for identifying the amount of nutrients that the user can ingest in association with the attribute of the user and the content of the meal or the content of the exercise habit.

4. The supplement proposal system according to claim 1,
wherein the answer reception unit receives answers to the user's drinking habits in addition to the answers to the question about the meal and the questions about the exercise habit, and
wherein the insufficient nutrient identification unit identifies the insufficient nutrients in accordance with the answer to each of the meal, the exercise habit, and the drinking habit.

5. The supplement proposal system according to claim 1,
wherein the supplement information storage unit stores a description of the supplement function against troubles for each of the troubles with respect to the supplement, and
wherein the supplement proposal unit outputs to the user a description of the supplement and the function corresponding to the received troubles, together with the selected supplement.

6. The supplement proposal system according to claim 1,
wherein the supplement information storage unit further stores a description of the supplement, and
the supplement proposal unit outputs the corresponding description to the user together with the selected supplement.

7. The supplement proposal system according to claim 1,
wherein the supplement information storage unit further stores allergy substance contained in the supplement,
the answer reception unit further receives an answer to the user's allergy, and
the supplement selection unit selects those not containing the allergy substance among the supplements containing the insufficient nutrients, in accordance with the answer regarding the allergy.

8. The supplement proposal system according to claim 2,
wherein the answer reception unit receives answers to the user's drinking habits in addition to the answers to the question about the meal and the questions about the exercise habit, and
wherein the insufficient nutrient identification unit identifies the insufficient nutrients in accordance with the answer to each of the meal, the exercise habit, and the drinking habit.

9. The supplement proposal system according to claim 3,
wherein the answer reception unit receives answers to the user's drinking habits in addition to the answers to the question about the meal and the questions about the exercise habit, and
wherein the insufficient nutrient identification unit identifies the insufficient nutrients in accordance with the answer to each of the meal, the exercise habit, and the drinking habit.

10. A supplement proposal server comprising:
a supplement information storage unit for storing nutrients contained in a supplement;
an answer reception unit for receiving an answer from a user to each of questions about meals ingested by the user and questions about the user's exercise habits;
an insufficient nutrient identifying unit for identifying nutrients lacking in the user in response to attribution of the user and the answer about each of the meal and the exercise habits from the user;
a supplement selection unit for selecting the supplements including the insufficient nutrients;
a supplement refining unit for refining the selected supplements; and
a supplement proposal unit for proposing the refined supplements to the user;
wherein the supplement information storage unit further stores a degree of recommendation of the supplement regarding the user's trouble,
wherein the answer reception unit further receives an input on the trouble and the degree of the trouble from the user, and
wherein the supplement refining unit reads the supplement corresponding to the trouble according to the degree of recommendation of the supplement, with reference to the supplement information storage unit, and also selects a predetermined range of numbers of supplements based on the degree of the trouble among the supplements containing the insufficient nutrients.

11. A supplement proposal program, wherein the supplement proposal program enables a supplement proposal terminal to function as:
a supplement information storage unit for storing nutrients contained in a supplement;
an answer reception unit for receiving an answer from a user to each of questions about meals ingested by the user and questions about the user's exercise habits;
an insufficient nutrient identifying unit for identifying nutrients lacking in the user in response to attribution of the user and the answer about each of the meal and the exercise habits from the user;
a supplement selection unit for selecting the supplements including the insufficient nutrients;
a supplement refining unit for refining the selected supplements; and
a supplement proposal unit for proposing the refined supplements to the user;
wherein the supplement information storage unit further stores a degree of recommendation of the supplement regarding the user's trouble,
wherein the answer reception unit further receives an input on the trouble and the degree of the trouble from the user, and
wherein the supplement refining unit reads the supplement corresponding to the trouble according to the degree of recommendation of the supplement, with reference to the supplement information storage unit, and also selects a predetermined range of numbers of supplements based on the degree of the trouble among the supplements containing the insufficient nutrients.
